(19) United States
(12) Patent Application Publication
(10) Pub. No.: US 2009/0187150 A1
(43) Pub. Date: Jul. 23, 2009

(54) METHOD AND APPARATUS FOR COVERING A NEEDLE OF A SYRINGE

(75) Inventors: Dominique Ferland, Laval (CA); Maurice Lavimodiere, Barrie (CA)

Correspondence Address: FASKEN MARTINEAU DUMOULIN, LLP, STOCK EXCHANGE TOWER, SUITE 3400, P.O. BOX 242, 800 PLACE VICTORIA, MONTREAL, QC, H4Z 1E9, CA

(73) Assignee: DOMREX PHARMA INC., Laval (CA)

(21) Appl. No.: 12/353,756

(22) Filed: Jan. 14, 2009

Related U.S. Application Data
(60) Provisional application No. 61/021,190, filed on Jan. 15, 2008.

(51) Int. Cl. A61M 5/32

(52) U.S. Cl. 604/197

(57) ABSTRACT

An apparatus for protecting a syringe after use, the syringe comprising a body and a needle, the apparatus comprising a hollow member for receiving the body of the syringe, the hollow member comprising attaching means for securing the hollow member to the body of the syringe and a protective member slidably mounted on the hollow member and slidable between a syringe operating position wherein the protective member covers at least one part of the body of the syringe and a permanent final position wherein the protective member cover said needle of the syringe. The apparatus advantageously reduces the risk associated with handling a syringe after use.

### RELATED APPLICATION

The present application claims priority of U.S. provisional patent application No. 61/021,190 filed on Jan. 15, 2008, the specification of which is incorporated herein by reference.

### FIELD OF THE INVENTION

The invention relates to protective devices. More precisely, the invention pertains to a method and apparatus for covering a needle of a syringe.

### BACKGROUND OF THE INVENTION

It is desirable to protect a user from the needle of a syringe after use for various reasons. For instance the product to be delivered using the syringe may be dangerous for the operator. Moreover, the needle may be contaminated after its use by blood or by body fluids from the patient which may contain virus for instance which can be transmitted by blood contact.

Since the operator has to often perform tasks quickly, the manipulation of a syringe can cause hazards.

Prior art devices for covering the needle of a syringe have many drawbacks.

For instance, the devices for covering the needle after use are not compatible with some syringe designs, but only with pre-filled glass syringes. This limits the potential use of the device.

In some other instances, some needle guard devices are fixed permanently on a specific syringe design and part of the device itself, this mean that it cannot be added to a regular syringe design. This again limits the potential use of the device.

There is therefore a need for an apparatus that will overcome at least one of the above-identified drawbacks.

### BRIEF SUMMARY

It is an object of the invention to provide an apparatus for covering a needle of a syringe that satisfies the above-mentioned need.

Accordingly, there is provided an apparatus for protecting a needle of a syringe after use, the syringe comprising a body, the apparatus comprising a hollow member for receiving the body of the syringe, the hollow member comprising attaching means for securing the hollow member to the body of the syringe, the apparatus comprising a protective member slidably mounted on the hollow member and slidable between a syringe operating position wherein the protective member covers at least one part of the body of the syringe and a permanent final position wherein the protective member cover the needle of said syringe.

The apparatus advantageously reduces the risk associated with handling a syringe after use.

Moreover, the apparatus may be mounted on existing syringes and does not require manufacturing a new syringe body for receiving the apparatus, which is of great advantage.

Furthermore, the apparatus may advantageously be used on syringes of various diameter sizes and can have various size and diameter suitable to cover a given range of syringes.

The apparatus may be mounted manually on a syringe body by a worker, working for a pharmaceutical company, thus reducing the need to change the manufacturing process, which is of great financial advantage.

Moreover, the apparatus may be mounted on a syringe by the end user, the healthcare medical professional with a regular protective cap on the needle, which is of great advantage for safety.

In one embodiment, the hollow member comprises an upper portion having a supporting surface adapted for resting on an upper surface of the body of the syringe.

This feature is of great advantage since the apparatus may be mounted on different syringes of different body lengths. Moreover, this feature may ease installation of the apparatus on a syringe. Also, because the apparatus is secured on the syringe body and not on the finger flange, the same design may fit different syringe body length that have the same diameter size and use the same needle length size.

In one embodiment, the attaching means comprise an adhesive substance and are provided with a colored element, preferably a blue element, in order to add a visual indication to a user that the adhesive is present, thereby improving safety of the apparatus, which is of great advantage.

In another embodiment, the attaching means of the hollow member comprise at least one flap securable to the body of the syringe.

In a further embodiment, the at least one flap comprises first and second flaps.

Still in a further embodiment, the first and second flaps are opposite to each other on the hollow member.

In one embodiment, the at least one flap comprises an inner side facing the body of the syringe, the inner side comprising an adhesive substance and a removable protecting layer covering the adhesive substance.

In a further embodiment, the adhesive substance comprises a double side tape adhesive.

In another embodiment, the hollow member comprises stop means cooperating with the protective member for preventing sliding of the protective member away from the hollow member.

In another embodiment, the protective member comprises at least one securing member cooperating with the hollow member so that the protective member is rigidly secured to the hollow member when the protective member is slid in the permanent final position.

In one embodiment, the at least one securing member comprises first and second securing members, each respectively comprising first and second flexible tongues.

In a further embodiment, each of the first and second tongues comprises a projecting member projecting inwards the protective member.

In still a further embodiment, the hollow member comprises an upper portion having an abutting element cooperating with the flexible tongues.

In another embodiment, the hollow member comprises a holding recess adapted for receiving the securing member therein in the syringe operating position.

In a further embodiment, the hollow member comprises first and second holding recesses, each being adapted for receiving a corresponding flexible tongue therein in the syringe operating position.

Still in a further embodiment, the first and second recesses are opposite to each other on the protective member.

In another embodiment, the first and second flaps are opposed to each other on the hollow member, the flaps and the recesses extending in an alternate configuration.

In another embodiment, the hollow member has an inner diameter corresponding to the outer diameter of the syringe.

In a further embodiment, the protective member has a predetermined shape so that the hollow member snugly fits therein.

In another embodiment, the body of the syringe has a predetermined outer diameter, the hollow member comprises an inner diameter corresponding to the outer diameter of the body of the syringe, and the protective member has an inner diameter corresponding to an outer diameter of the hollow member for providing a snugly fit between the hollow member and the protective member.

In one embodiment, the hollow member is made of a material selected from a group consisting of thermoplastic polymer and plastic base substance.

In a further embodiment, the hollow member is made of polyethylene.

In one embodiment, the protective member is made of a material selected from a group consisting of thermoplastic polymer and plastic base substance.

In a further embodiment, the protective member is made of polycarbonate.

It is another object of the invention to provide a method for protecting a needle of a syringe after use.

Accordingly, there is provided a method for protecting a needle of a syringe after use, the syringe comprising a body, the method comprising providing an apparatus comprising a hollow member for receiving the body of the syringe, the hollow member comprising attaching means for securing the hollow member to the body of the syringe, and a protective member slidably mounted on the hollow member and slidable between a syringe operating position wherein the protective member covers at least one part of the body of the syringe and a permanent final position wherein the protective member cover the needle of the syringe; inserting the syringe into the hollow member of the apparatus; securing the attaching means of the hollow member to the body of the syringe; and after use of said syringe, sliding the protective member in the permanent final position wherein the protective member cover the needle of the syringe.

### BRIEF DESCRIPTION OF THE DRAWINGS

These and other objects and advantages of the invention will become apparent upon reading the detailed description and upon referring to the drawings in which:

FIG. 1 is a 3D perspective view of an embodiment of an apparatus for covering a needle of a syringe wherein the syringe has been inserted in the apparatus and further wherein the apparatus comprises two flaps.

FIG. 2 is a front elevation view of the apparatus disclosed in FIG. 1.

FIG. 3 is a rear elevation view of the apparatus disclosed in FIG. 1.

FIG. 4 is a right elevation view of the apparatus disclosed in FIG. 1.

FIG. 5 is a left elevation view of the apparatus disclosed in FIG. 1.

FIG. 6 is a top view of the apparatus disclosed in FIG. 1.

FIG. 7 is a bottom view of the apparatus disclosed in FIG. 1.

FIG. 8 is a 3D perspective view of an embodiment of an apparatus for covering a needle of a syringe wherein the apparatus has been secured to the body of the syringe using the attaching means.

FIG. 9 is a front elevation view of the apparatus disclosed in FIG. 8.

FIG. 10 is a rear elevation view of the apparatus disclosed in FIG. 8.

FIG. 11 is a right elevation view of the apparatus disclosed in FIG. 8.

FIG. 12 is a left elevation view of the apparatus disclosed in FIG. 8.

FIG. 13 is a top view of the apparatus disclosed in FIG. 8.

FIG. 14 is a bottom view of the apparatus disclosed in FIG. 8.

FIG. 15 is a 3D perspective view of an embodiment of an apparatus for covering a needle of a syringe wherein the protective member of the apparatus has been engaged in a permanent position in which it covers the needle.

FIG. 16 is a front elevation view of the apparatus disclosed in FIG. 15.

FIG. 17 is a rear elevation view of the apparatus disclosed in FIG. 15.

FIG. 18 is a right elevation view of the apparatus disclosed in FIG. 15.

FIG. 19 is a left elevation view of the apparatus disclosed in FIG. 15.

FIG. 20 is a top view of the apparatus disclosed in FIG. 15.

FIG. 21 is a bottom view of the apparatus disclosed in FIG. 15.

FIG. 22 is a first exploded view which shows one embodiment of the apparatus for covering the needle of a syringe wherein the attaching means have been engaged.

FIG. 23 is a second exploded view which shows one embodiment of the apparatus for covering the needle of a syringe wherein the attaching means have not been engaged.

FIG. 24 is a cross-sectional view of one embodiment of the apparatus taken along lines 24-24 of FIG. 6.

FIG. 25 is a cross-sectional view of one embodiment of the apparatus taken along lines 25-25 of FIG. 6.

FIG. 26 is a cross-sectional view of one embodiment of the apparatus taken along lines 26-26 of FIG. 13.

FIG. 27 is a cross-sectional view of one embodiment of the apparatus taken along lines 27-27 of FIG. 20.

FIG. 28 is a cross-sectional view of one embodiment of the apparatus taken along lines 28-28 of FIG. 20.

FIG. 29 is a 3D perspective view of another embodiment of an apparatus for covering a needle of a syringe wherein the syringe has been inserted in the apparatus and further wherein the apparatus comprises a single flap.

FIG. 30 is a front elevation view of the apparatus for covering a needle of a syringe disclosed in FIG. 29.

FIG. 31 is a rear elevation view of the apparatus for covering a needle of a syringe disclosed in FIG. 29.

FIG. 32 is a right elevation view of the apparatus for covering a needle of a syringe disclosed in FIG. 29.

FIG. 33 is a left elevation view of the apparatus for covering a needle of a syringe disclosed in FIG. 29.

FIG. 34 is a top view of the apparatus for covering a needle of a syringe disclosed in FIG. 29.

FIG. 35 is a bottom view of the apparatus for covering a needle of a syringe disclosed in FIG. 29.

FIG. 36 is an exploded view of an embodiment of an apparatus for covering a needle of a syringe wherein the apparatus comprises a single flap.

FIG. 37 is a 3D perspective view of an embodiment of an apparatus for covering a needle of a syringe wherein the apparatus comprises a single flap and further wherein the protective member of the apparatus has been pulled.

FIG. 38 is a front elevation view of the apparatus for covering a needle of a syringe disclosed in FIG. 37.

FIG. 39 is a rear elevation view of the apparatus for covering a needle of a syringe disclosed in FIG. 37.

FIG. 40 is a right elevation view of the apparatus for covering a needle of a syringe disclosed in FIG. 37.

FIG. 41 is a left elevation view of the apparatus for covering a needle of a syringe disclosed in FIG. 37.

FIG. 42 is a top view of the apparatus for covering a needle of a syringe disclosed in FIG. 37.

FIG. 43 is a bottom view of the apparatus for covering a needle of a syringe disclosed in FIG. 37.

FIG. 44 is a cross-sectional view of one embodiment of the apparatus taken along lines 44-44 of FIG. 34.

FIG. 45 is a cross-sectional view of one embodiment of the apparatus taken along lines 45-45 of FIG. 34.

FIG. 46 is a cross-sectional view of one embodiment of the apparatus taken along lines 46-46 of FIG. 42.

FIG. 47 is a cross-sectional view of one embodiment of the apparatus taken along lines 47-47 of FIG. 42.

FIG. 48 is a 3D perspective view of another embodiment of an apparatus for covering a needle of a syringe wherein the syringe has been inserted in the apparatus and further wherein the apparatus comprises a single flap.

FIG. 49 is a front elevation view of the apparatus disclosed in FIG. 48.

FIG. 50 is a rear elevation view of the apparatus disclosed in FIG. 48.

FIG. 51 is a right elevation view of the apparatus disclosed in FIG. 48.

FIG. 52 is a left elevation view of the apparatus disclosed in FIG. 48.

FIG. 53 is a top view of the apparatus disclosed in FIG. 48.

FIG. 54 is a bottom view of the apparatus disclosed in FIG. 48.

FIG. 55 is a 3D perspective view of an embodiment of an apparatus for covering a needle of a syringe wherein the protective member of the apparatus has been pulled.

FIG. 56 is a front elevation view of the apparatus disclosed in FIG. 55.

FIG. 57 is a rear elevation view of the apparatus disclosed in FIG. 55.

FIG. 58 is a right elevation view of the apparatus disclosed in FIG. 55.

FIG. 59 is a left elevation view of the apparatus disclosed in FIG. 55.

FIG. 60 is a top view of the apparatus disclosed in FIG. 55.

FIG. 61 is a bottom view of the apparatus disclosed in FIG. 55.

FIG. 62 is an exploded view which shows one embodiment of the apparatus for covering the needle of a syringe wherein the attaching means have been engaged.

FIG. 63 is a cross-sectional view of one embodiment of the apparatus taken along lines 63-63 of FIG. 53.

FIG. 64 is a cross-sectional view of one embodiment of the apparatus taken along lines 64-64 of FIG. 53.

FIG. 65 is a cross-sectional view of one embodiment of the apparatus taken along lines 65-65 of FIG. 60; and

FIG. 66 is a cross-sectional view of one embodiment of the apparatus taken along lines 66-66 of FIG. 60.

Further details of the invention and its advantages will be apparent from the detailed description included below.

### DETAILED DESCRIPTION

In the following description of the embodiments, references to the accompanying drawings are by way of illustration of an example by which the invention may be practiced. It will be understood that other embodiments may be made without departing from the scope of the invention disclosed.

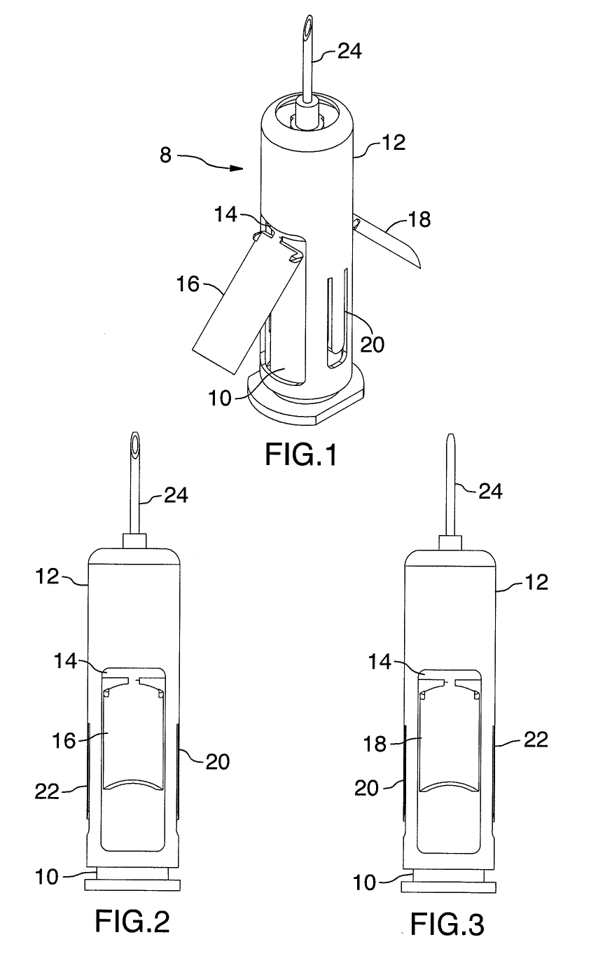

Now referring to FIG. 1, there is shown an embodiment of an apparatus 8 for covering a needle 24 of a syringe 10 after use.

More precisely, the apparatus 8 comprises a hollow member 14 for receiving the body of the syringe 10. The hollow member 14 comprises attaching means, one embodiment of which is first flap 16 and second flap 18 for securing the hollow member 14 to the body of the syringe 10.

In the embodiment disclosed, the attaching means comprises flaps 16 and 18 which are opposed to each other. In others words, flaps 16 and 18 extend at 180 degrees from each other. It will however be appreciated by the skilled addressee that the attaching means may comprise a single flap or alternatively a plurality of flaps as shown further below. The skilled addressee will also appreciate that any other means adapted for securing the hollow member 14 to the body of the syringe may also be considered.

In one embodiment, the hollow member 14 is made of Polyethylene such as Dow LDPE 722 manufactured by Dow Plastics. Alternatively, various other types of thermoplastic polymer or plastic base substance may be used.

Still in this embodiment, each of the flaps 16 and 18 is covered with an adhesive substance protected, before its use, by a piece of material to be removed by an operator before use. In other words, each of the flaps 16 and 18 has an inner side facing the body of the syringe 10, the inner side comprising an adhesive substance such as an adhesive layer and a removable protecting layer covering the adhesive substance. It has been contemplated that the adhesive layer may be a double side tape adhesive manufacturer by the 3M Company. Alternatively, various other types of adhesive substance may be used.

As shown in FIG. 1, the apparatus 8 further comprises a protective member 12, or cover, which is slidably mounted on the hollow member 14. The protective member 12 is slidable between a syringe operating position wherein the protective member 12 covers at least one part of the body of the syringe 10 and a permanent final position wherein the protective member 12 covers the needle 24 of the syringe 10.

It will be appreciated by the skilled addressee that in the embodiment disclosed in FIG. 1, the protective member 12 is in the syringe operating position and that it therefore does not cover the needle 24 of the syringe 10. In other words, in this position, the needle 24 is accessible as usually for its intended purpose.

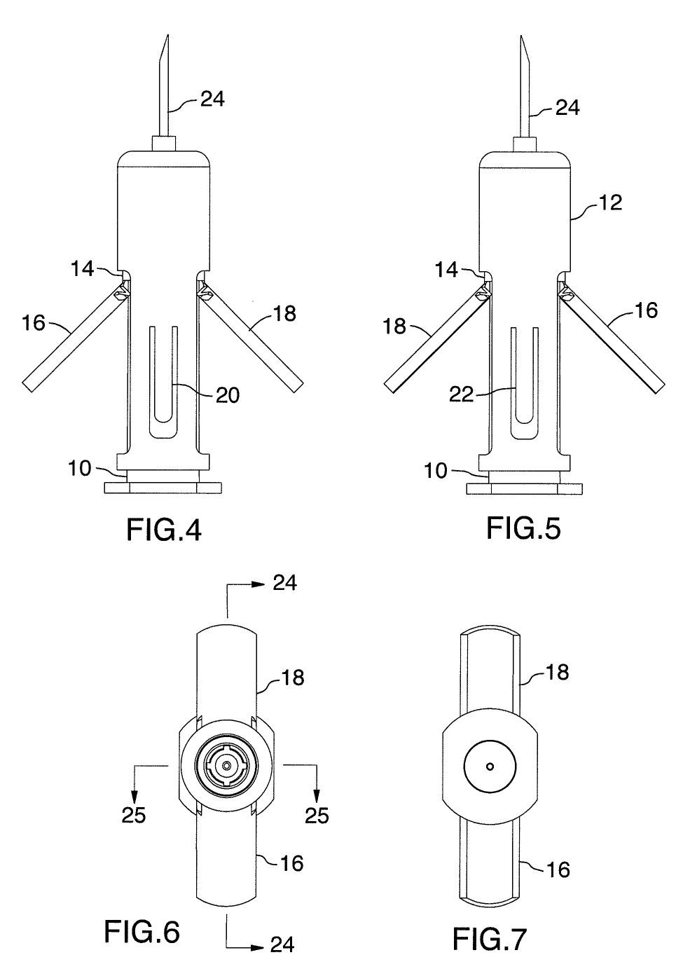

Now referring to FIG. 2, there is shown a front side elevation view of the apparatus 8 for covering a needle 24 of the syringe 10 disclosed in FIG. 1. FIG. 3 shows a rear side elevation view of the apparatus 8 for covering the needle 24 of a syringe 10 disclosed in FIG. 1 while FIG. 4 shows a right side elevation view of the apparatus 8 for covering the needle 24 of the syringe 10 disclosed in FIG. 1 and FIG. 5 shows a left side elevation view of the apparatus 8 disclosed in FIG. 1.

FIG. 6 shows a top view of the apparatus 8 for covering the needle 24 of a syringe 10 disclosed in FIG. 1 while FIG. 7 shows a bottom view of the apparatus 8 for covering the needle 24 of the syringe 10 disclosed in FIG. 1.

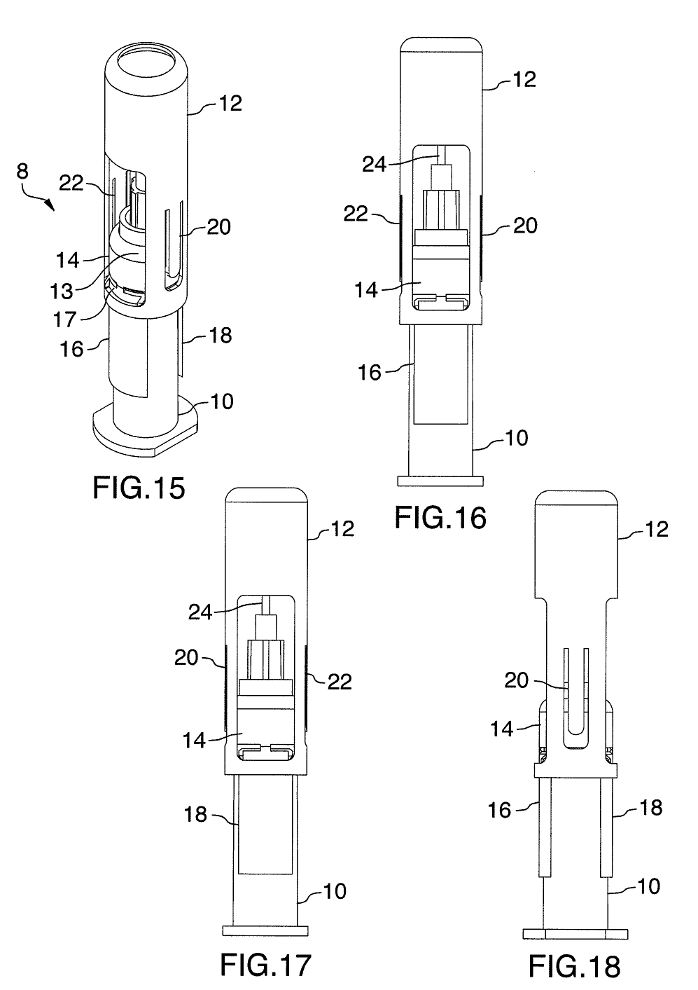

FIG. 15, which will be described in more details thereinafter, shows an embodiment of the apparatus 8 wherein the protective member 12 has been slid in the permanent final position. As shown, the protective member 12 covers the needle 24, thereby protecting any operator from the needle 24 of the syringe 10.

It will be appreciated that in one embodiment the protective member 12 will be kept in the permanent final position using first and second securing members 20 and 22 located on the protective member 12, these securing members 20, 22 cooperating with the hollow member 14, as shown in FIG. 15.

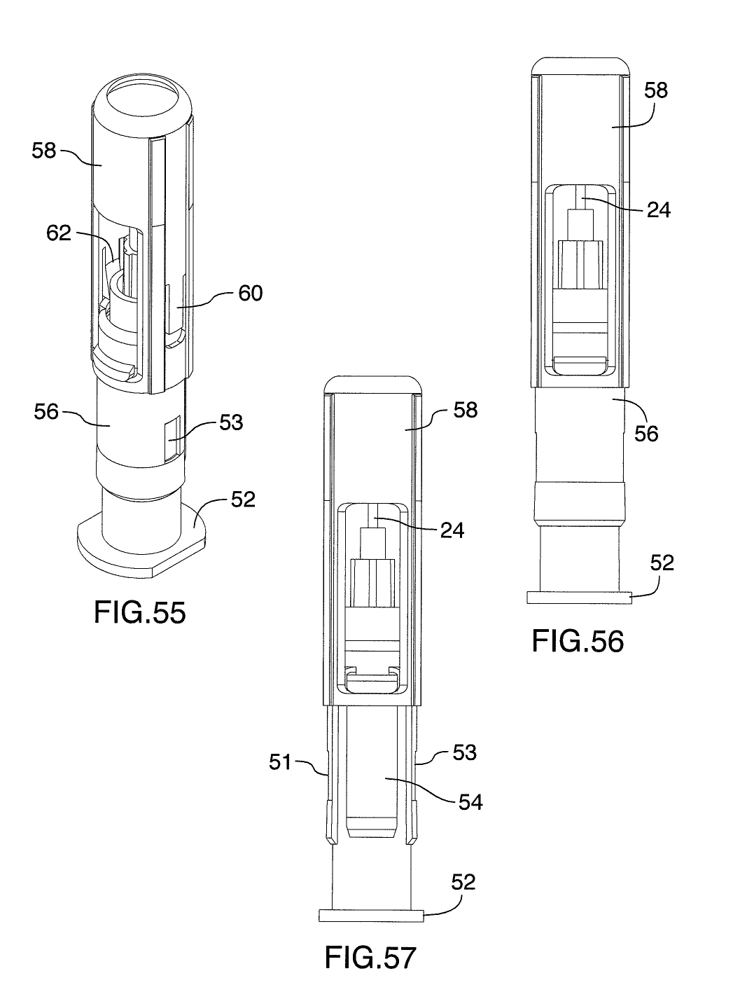

In the illustrated embodiment, the first and second securing members respectively comprise first and second flexible tongues 20, 22, each comprising a projecting member projecting inwards the protective member 12. The hollow member 14 comprises an upper portion having an abutting element 13 which cooperates with the flexible tongues 20, 22. As shown in FIG. 15 and also in FIG. 55 which will be described further, once the protective member 12 has been slid in the permanent final position, the flexible tongues 20, 22 are engaged with the abutting element 13 of the upper portion of the hollow member 14 and prevent the operator from moving the protective member in the operating position.

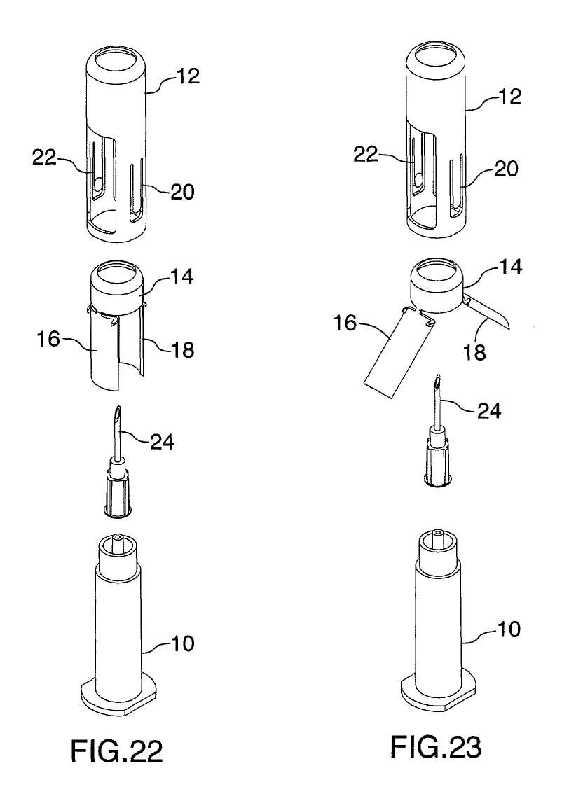

In the embodiment illustrated in FIG. 15, first and second tongues 20, 22 opposite to each other are used but the skilled addressee will appreciate that various other embodiments may be used for maintaining the protective member 12 in the permanent final position. In this embodiment, flaps 16, 18 extend at 180 degrees from each other and are arranged in an alternate pattern with the tongues 20, 22. Moreover, each of the first and second tongues comprises a projecting member projecting inwards the protective member and cooperating with the abutting element 13, as better shown in FIG. 22.

The skilled addressee will appreciate that once slid, the protective member 12 will rest in the permanent final position thanks to stop means arranged on the hollow member 14. Indeed, in the illustrated embodiment, the upper portion of the flaps 16, 18 act as stop means 17 preventing the protective member 12 from sliding away from the hollow member 14 while the flexible tongues 20, 22 prevent the protective member from sliding in the original position. In other words, the disclosed arrangement enables to trap the protective member 12 in the final permanent position once it is slid.

In the embodiment of FIG. 15, tongues 20, 22 are free to slide in the hollow spaces created between the flaps 16, 18 but the skilled addressee will appreciate that alternate embodiments may be considered, as further explained below with reference to FIG. 55.

In one embodiment, the protective member 12 is made of polycarbonate, such as Makrolon® 2658 manufactured by Bayer Material Science LLC. Alternatively, various other types of thermoplastic polymer or plastic base substance may be used.

It will be appreciated that the apparatus 8 for covering the needle 24 of a syringe 10 may be packaged in various configurations. In one embodiment, the apparatus may be packaged in a transparent sterilized packaging with or without one paper side.

In order to use the apparatus 8 with a given syringe and according to one embodiment, an operator will first remove the apparatus from its package, if any.

The operator will then insert the given syringe in the apparatus 8. The operator will then secure the apparatus 8 to the body of the syringe 10. In one embodiment, this is performed by securing the attaching means of the hollow member 14 of the apparatus 8 to the body of the syringe 10.

In one embodiment, the hollow member 14 may comprise an upper portion having a supporting surface adapted for resting on an upper surface of the body of the syringe 10. This feature is of great advantage since it simplifies the insertion of the syringe 10 in the apparatus 8. Indeed, for mounting the apparatus 8 on a syringe 10, the user has to insert the syringe 10 in the apparatus 8 and push the syringe 10 therein until the upper surface of the body of the syringe abuts against the supporting surface of the upper portion of the hollow member 14 of the apparatus 8. Then the user can secure the hollow member 14 to the syringe 10, as previously explained. Moreover, as it will be more clearly understood upon reading of the present description, this feature is particularly advantageous since it may allow the insertion of a syringe whose body has a length comprised in a predetermined range. In other words, this arrangement may be used with different syringes of different body lengths.

The operator will then use the syringe 10 and after its use, the operator will hold the body of the syringe 10 or the hollow member 14 using one hand in one embodiment and pull the protective member 12 of the apparatus 8 using the other hand until the permanent final position in reached, in which the protective member 12 fully covers the needle 24 of the apparatus 8.

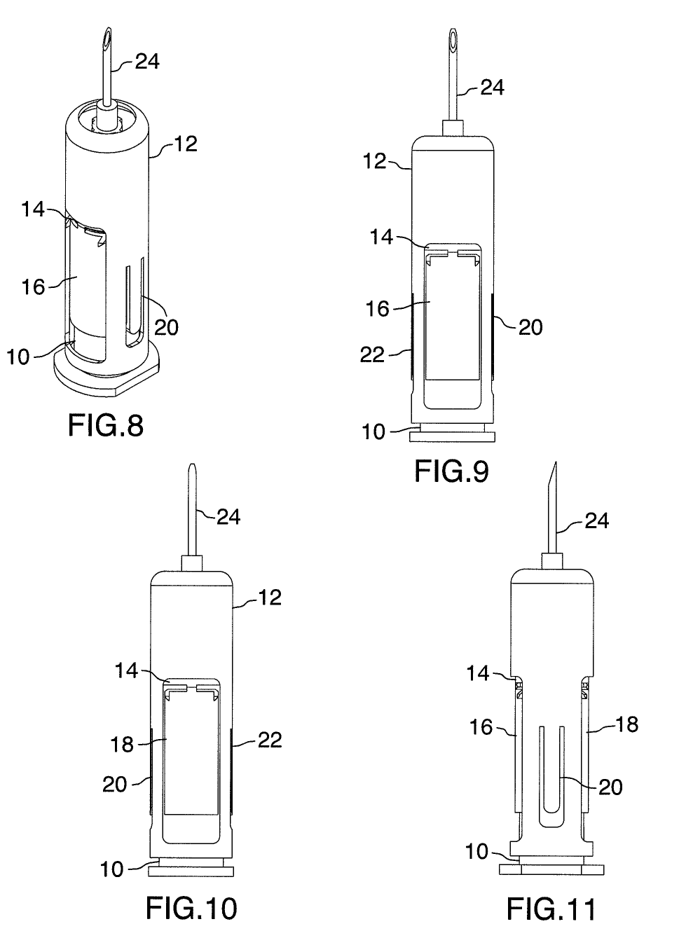

Now referring to FIG. 8, there is shown an embodiment of the apparatus 8 for covering the needle 24 of the syringe 10 after use.

In this embodiment, the apparatus 8 has been secured to the body of the syringe 10. It will be appreciated by the skilled addressee that this is performed prior using the syringe 10 as explained above. It will also be appreciated by the skilled addressee that the apparatus may be mounted on the syringe, whether its needle is provided with a cap or not. As explained above and in one embodiment, the apparatus 8 is secured to the body of the syringe 10 using the attaching means, one embodiment of which is flaps 16 and 18. More precisely, this is performed by pushing each of the flaps 16 and 18 against the body of the syringe 10. It will be appreciated by the skilled addressee that in one embodiment the corresponding surface of each of the flaps 16 and 18 surrounding the body of the syringe 10 is provided with an adhesive substance such as an adhesive layer as explained above. In such embodiment, the operator may have first to remove a protective layer covering the adhesive substance prior pushing each of the flaps 16 and 18 against the body of the syringe 10.

In one embodiment, the flaps 16 and 18 are pushed against the body of the syringe 10 using the fingers of the operator.

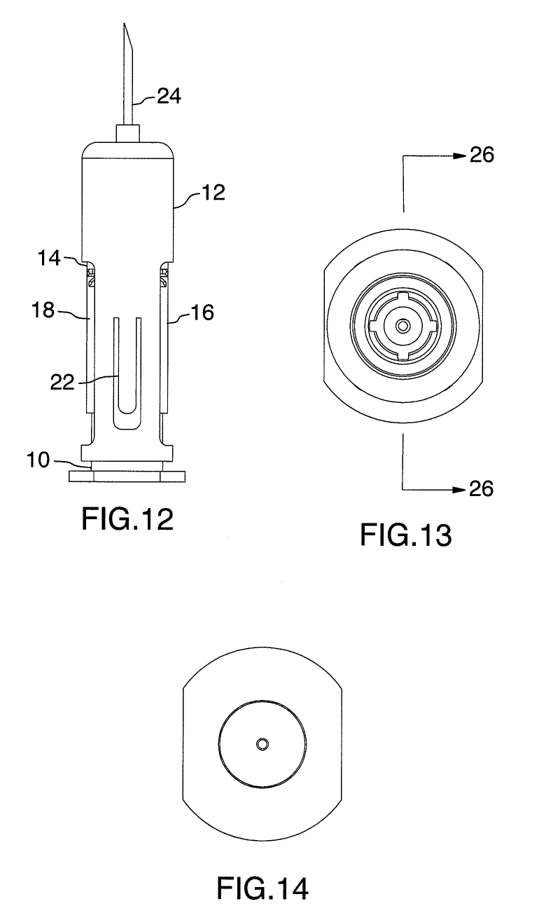

Now referring to FIG. 9, there is shown a front side elevation view of the apparatus 8 for covering the needle 24 of the syringe 10 disclosed in FIG. 8. FIG. 10 shows a rear side elevation view of the apparatus 8 for covering the needle 24 of the syringe 10 disclosed in FIG. 8. FIG. 11 shows a right side elevation view of the apparatus 8 for covering the needle 24 of the syringe 10 disclosed in FIG. 8 while FIG. 12 shows a left side elevation view of the apparatus 8 for covering the needle 24 of the syringe 10 disclosed in FIG. 8.

FIG. 13 shows a top view of the apparatus 8 for covering the needle 24 of the syringe 10 disclosed in FIG. 8 while FIG. 14 shows a bottom view of the apparatus 8 for covering the needle 24 of the syringe 10 disclosed in FIG. 8.

Now referring to FIG. 15, there is shown an embodiment of the apparatus 8 for covering the needle 24 of the syringe 10 after the use of the syringe 10.

As shown in FIG. 15, the protective member 12 of the apparatus 8 has been pulled and is now in the permanent final position. In one embodiment, the protective member 12 has been pulled by the operator. It will be appreciated by the skilled addressee that, as previously mentioned, because the protective member 12 of the apparatus 8 is in the permanent final position, any operator is now protected from the needle 24 of the syringe 10.

Now referring to FIG. 16, there is shown a front side elevation view of the apparatus 8, shown in FIG. 15, for covering the needle 24 of the syringe 10 after the use of the syringe 10. FIG. 17 shows a rear side elevation view of the apparatus 8, shown in FIG. 15, for covering the needle 24 of the syringe 10 after the use of the syringe 10.

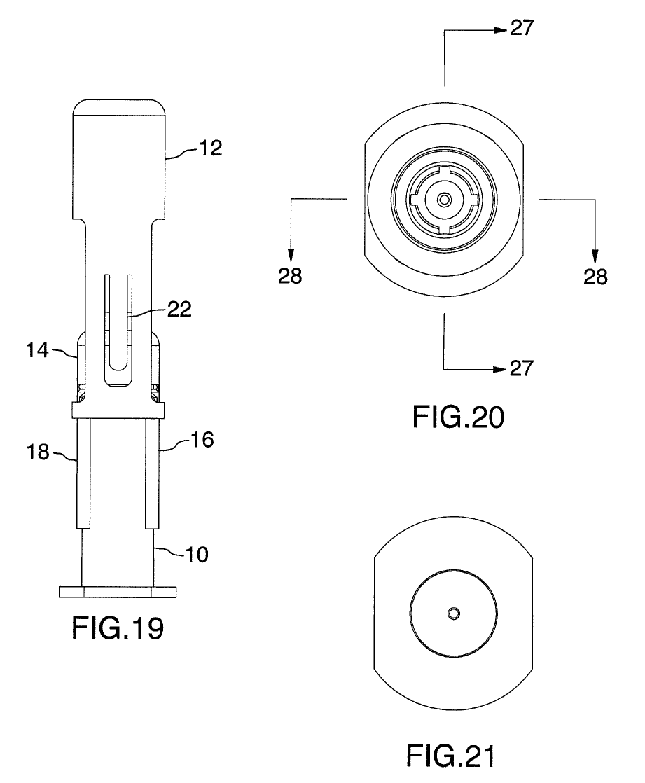

FIG. 18 shows a right side elevation view of the apparatus 8, shown in FIG. 15, for covering the needle 24 of the syringe 10 after the use of the syringe 10. FIG. 19 shows a left side elevation view of the apparatus 8, shown in FIG. 15, for covering the needle 24 of the syringe 10 after the use of the syringe 10.

FIG. 20 shows a top view of the apparatus 8, shown in FIG. 15, for covering the needle 24 of the syringe 10 after the use of the syringe 10.

FIG. 21 shows a bottom view of the apparatus 8, shown in FIG. 15, for covering the needle 24 of the syringe 10 after the use of the syringe 10.

Now referring to FIG. 22, there is shown a first exploded view which shows one embodiment of the apparatus 8 for covering the needle 24 of the syringe 10 wherein the flaps 16 and 18 are in a vertical position, i.e. the flaps 16, 18 have been pushed against the body of the syringe 10.

FIG. 23 shows a second exploded view which shows one embodiment of the apparatus 8 for covering the needle 24 of the syringe 10 wherein the flaps 16 and 18 are not in a vertical position, i.e. the flaps 16, 18 are in their initial position.

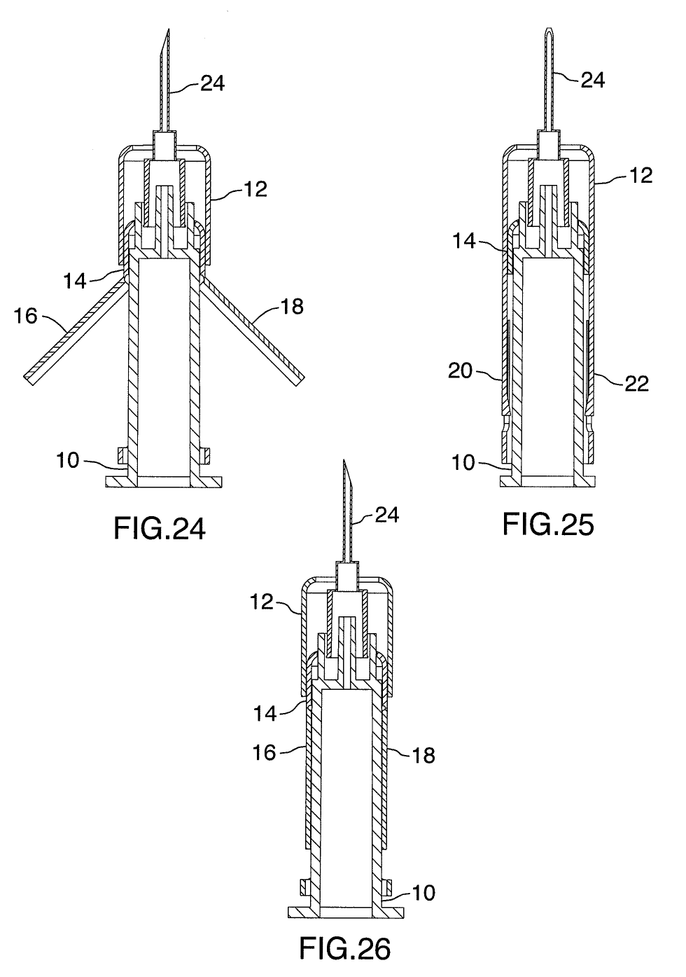

FIG. 24 shows a cross-sectional view, taken along lines 24-24 of FIG. 6, of one embodiment of the apparatus 8 for covering the needle 24 of the syringe 10 while FIG. 25 shows a cross-sectional view, taken along lines 25-25 of FIG. 6, of one embodiment of the apparatus 8 for covering the needle 24 of the syringe 10.

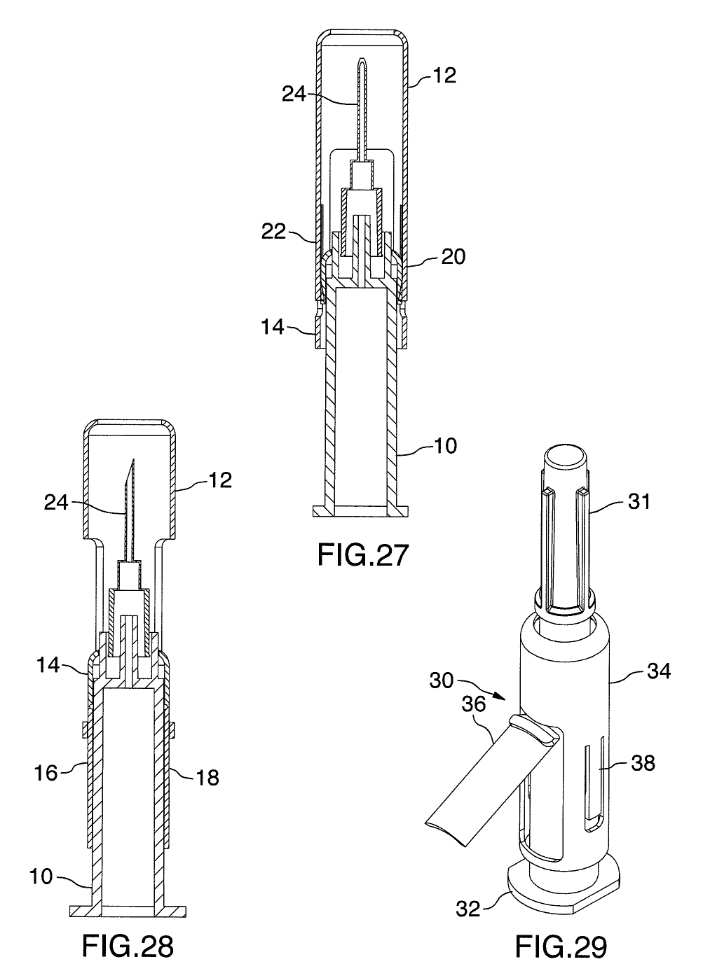

FIG. 26 shows a cross-sectional view, taken along lines 26-26 of FIG. 13, of one embodiment of the apparatus 8 for covering the needle 24 of the syringe 10 while FIG. 27 shows a cross-sectional view, taken along lines 27-27 of FIG. 20, of one embodiment of the apparatus 8 for covering the needle 24 of the syringe 10.

FIG. 28 is a cross-sectional view, taken along lines 28-28 of FIG. 20, of one embodiment of the apparatus 8 for covering the needle 24 of the syringe 10.

Now referring to FIG. 29, there is shown another embodiment of an apparatus 30 for covering a needle 42 of a syringe 32 after use.

In this embodiment, the attaching means of the hollow member 39 of the apparatus 30 comprises a single flap 36 which may comprises an adhesive substance on its inner side and a protective layer extending thereon, as previously described. The protective member 24 of the apparatus 30 comprises securing members 38 and 40 which may cooperate with the upper portion of the hollow member 39 to rigidly secure the protective member 34 in the final permanent position once slid.

While it has been disclosed in FIG. 29 that a cap 31 is covering the needle 42 of the syringe 32, the skilled addressee will appreciate that this cap 31 is removed for use.

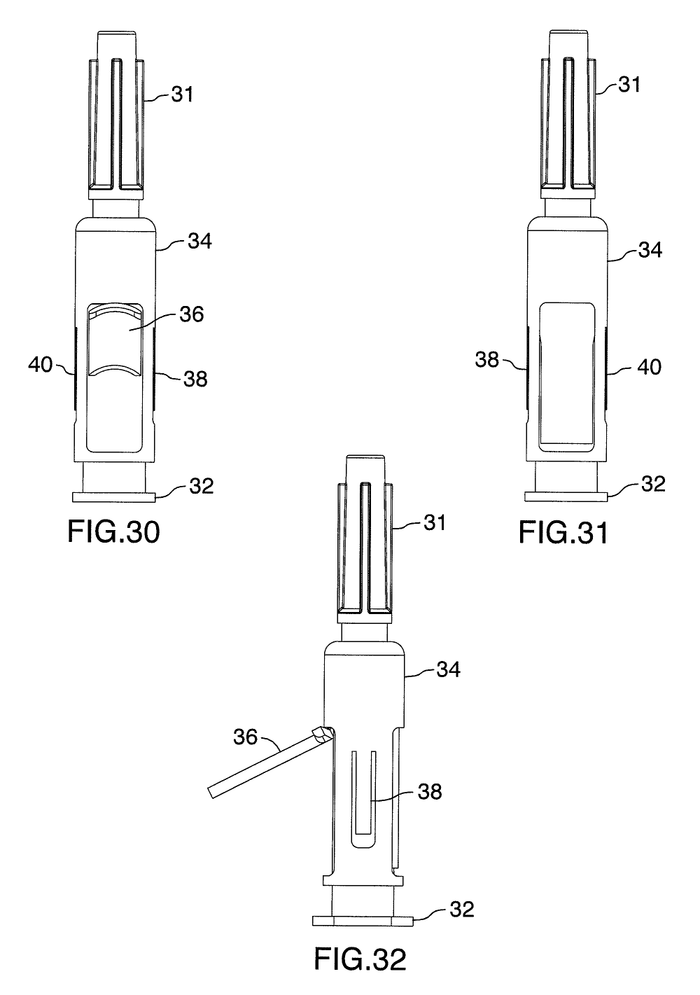

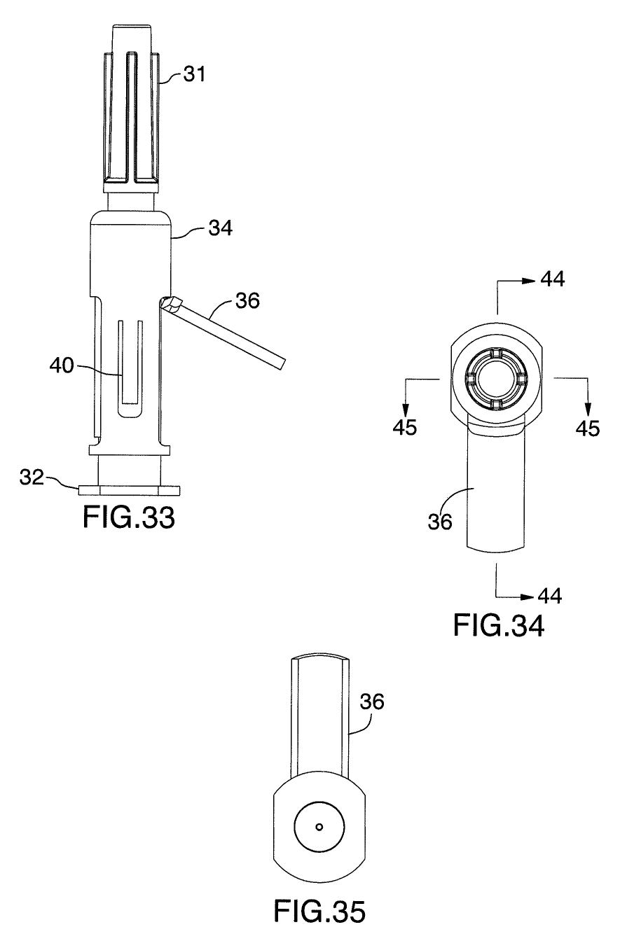

FIG. 30 shows a front elevation view of the apparatus 30 for covering the needle 42 of the syringe 32 after use while FIG. 31 shows a rear elevation view of the apparatus 30 for covering the needle 42 of the syringe 32, FIG. 32 shows a right elevation view of the apparatus 30 for covering the needle 42 of the syringe 32 and FIG. 33 shows a left elevation view of the apparatus 30 for covering the needle 42 of the syringe 32. FIGS. 34 and 35 respectively show top and bottom view of the apparatus 30 for covering the needle 42 of the syringe 32.

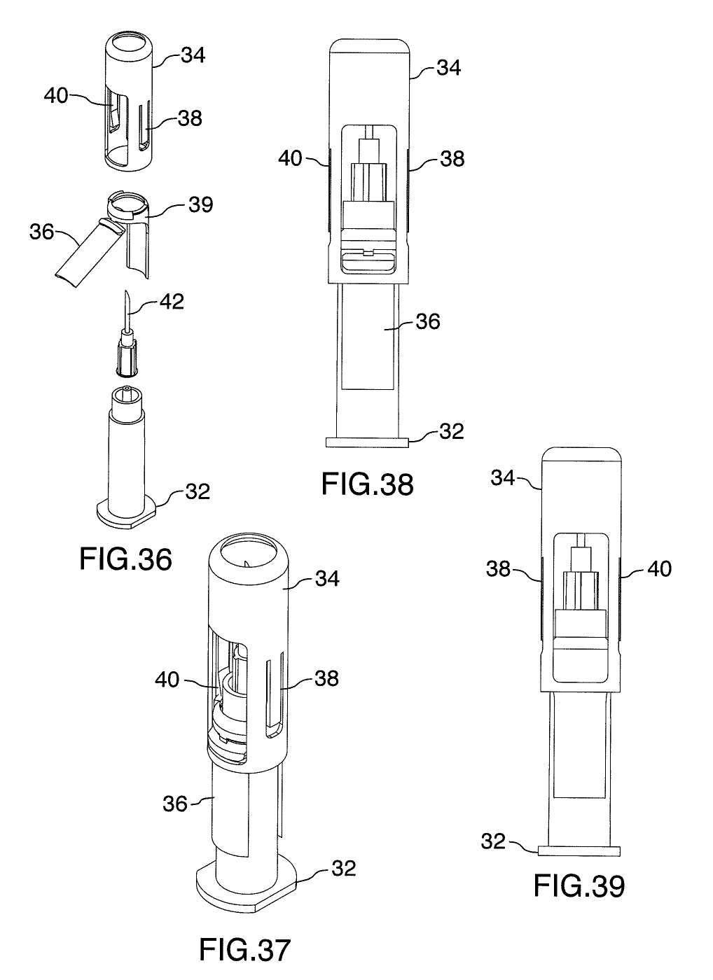

FIG. 36 shows an exploded view of the apparatus 30 for covering the needle 42 of the syringe 32 after use. As clearly shown, the hollow member 39 comprises a single flap 36.

FIG. 37 shows one embodiment of the apparatus 30 for covering the needle 42 of the syringe 32 after use wherein the flap 36 has been secured to the body of the syringe 32 and further wherein the protective member 34 has been pulled to thereby protect the needle 42 of the syringe 32.

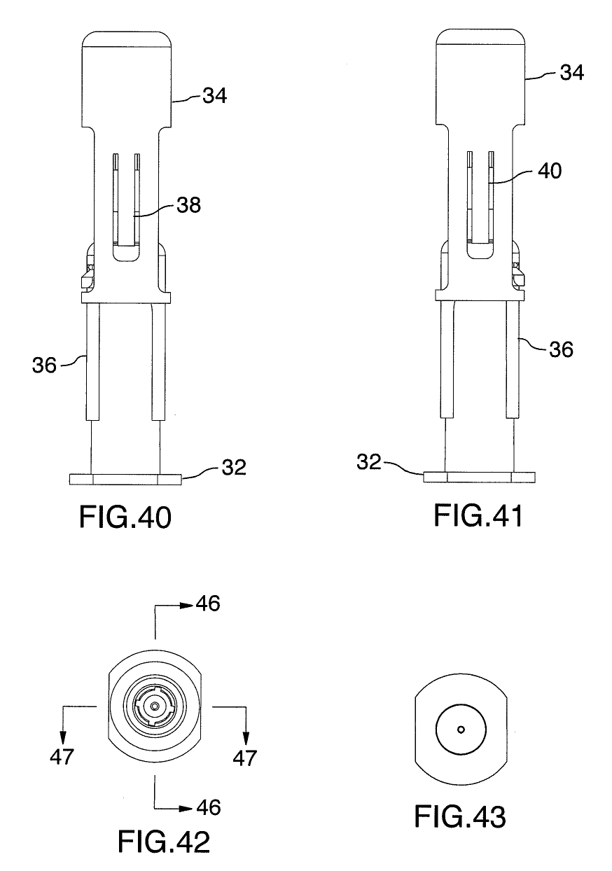

FIG. 38 shows a front elevation view of the apparatus 30 shown in FIG. 37 while FIG. 39 shows a rear elevation view of the apparatus 30 shown in FIG. 37, FIG. 40 shows a right elevation view of the apparatus 30 shown in FIG. 37 and FIG. 41 shows a left elevation view of the apparatus 30 shown in FIG. 37. Finally FIGS. 42 and 43 shows respectively a top and bottom view of the apparatus 30 shown in FIG. 37.

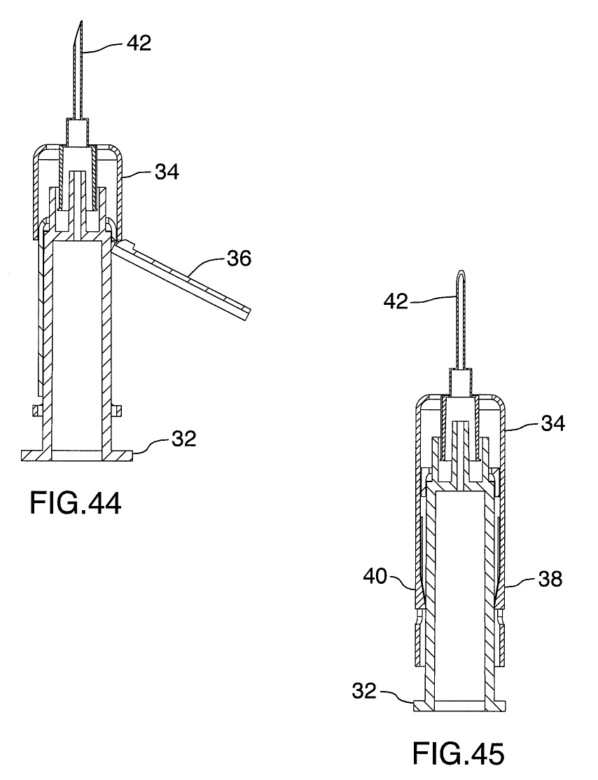

FIG. 44 shows a cross-sectional view, taken along lines 44-44, of the apparatus 30 shown in FIG. 34.

FIG. 45 shows a cross-sectional view, taken along lines 45-45, of the apparatus 30 shown in FIG. 34.

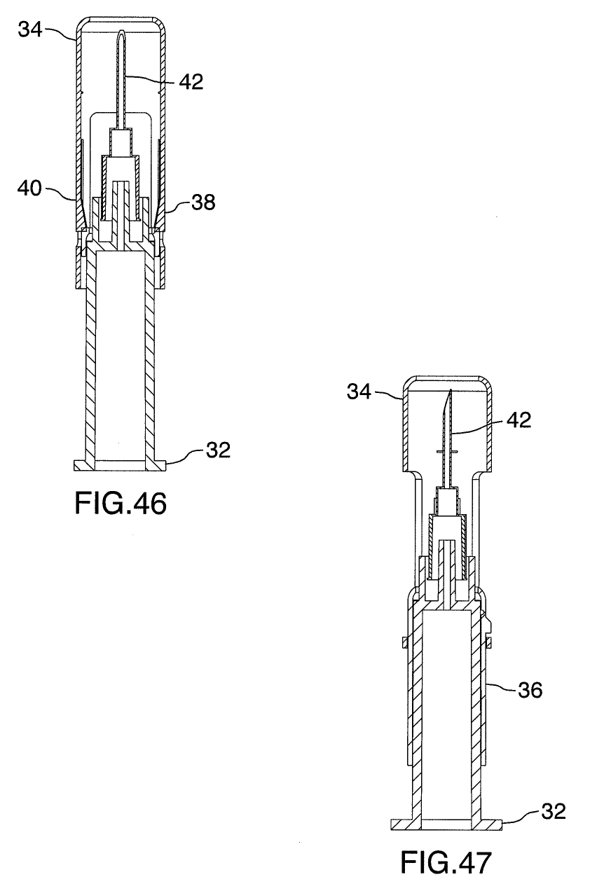

FIG. 46 shows a cross-sectional view, taken along lines 46-46, of the apparatus 30 shown in FIG. 42.

FIG. 47 shows a cross-sectional view, taken along lines 47-47, of the apparatus 30 shown in FIG. 42.

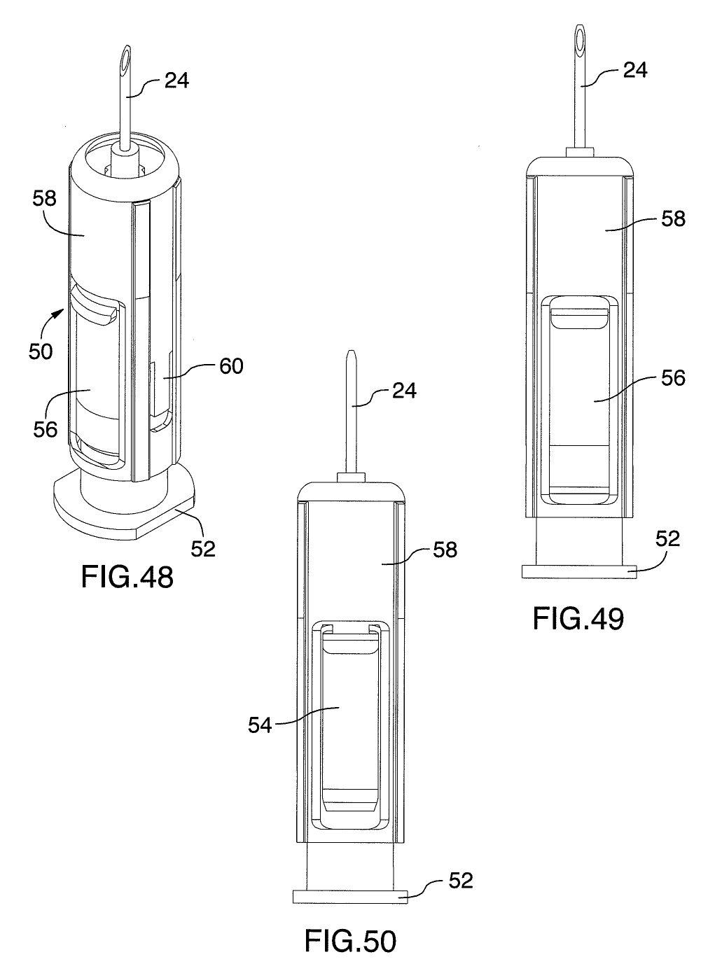

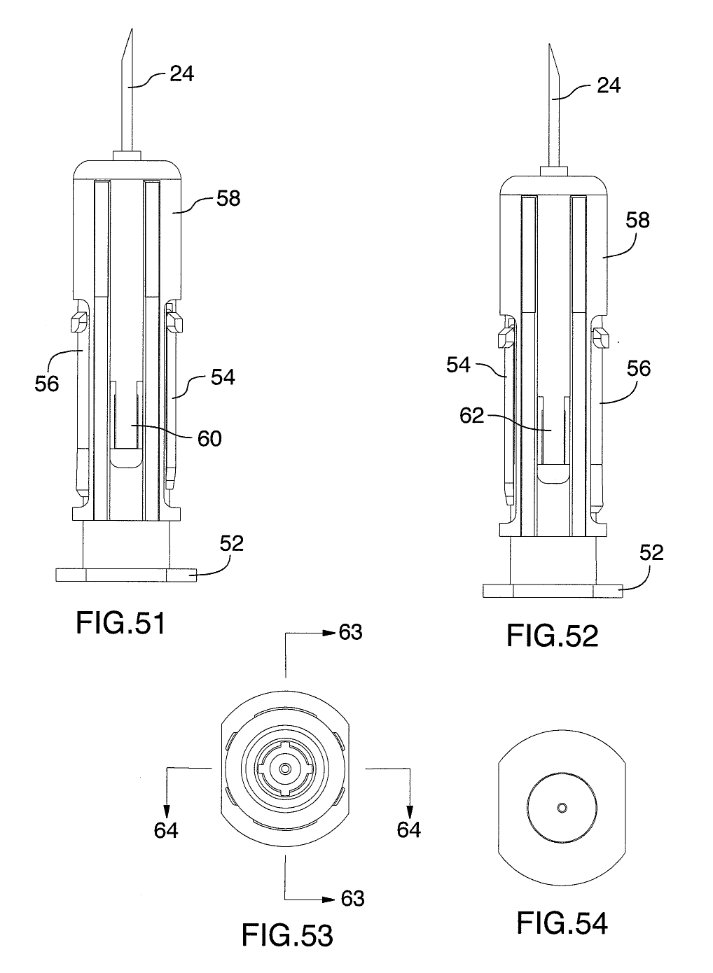

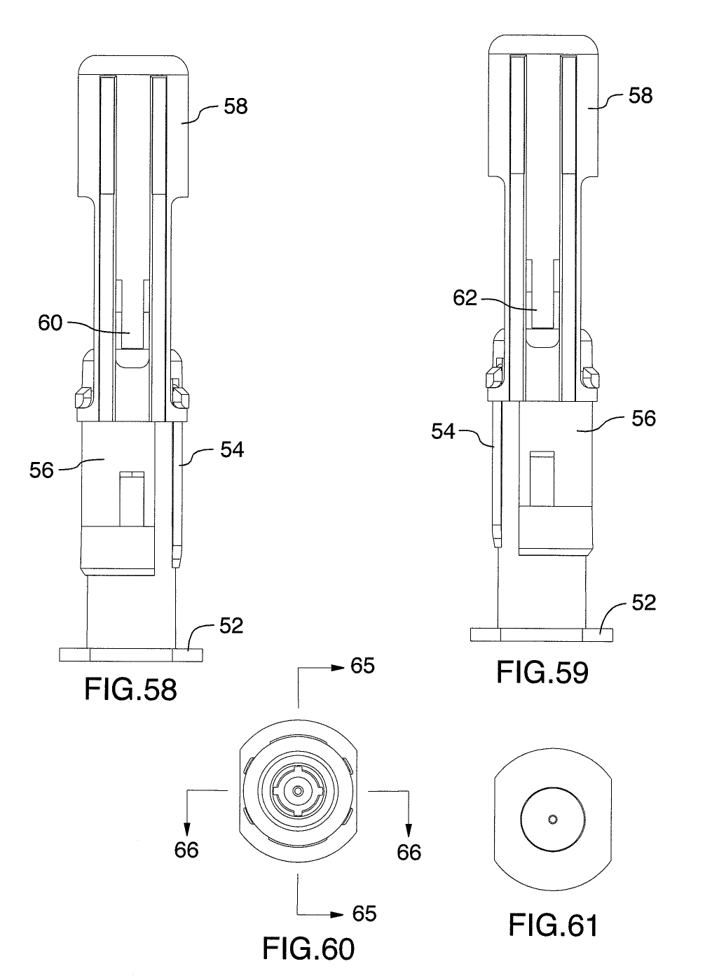

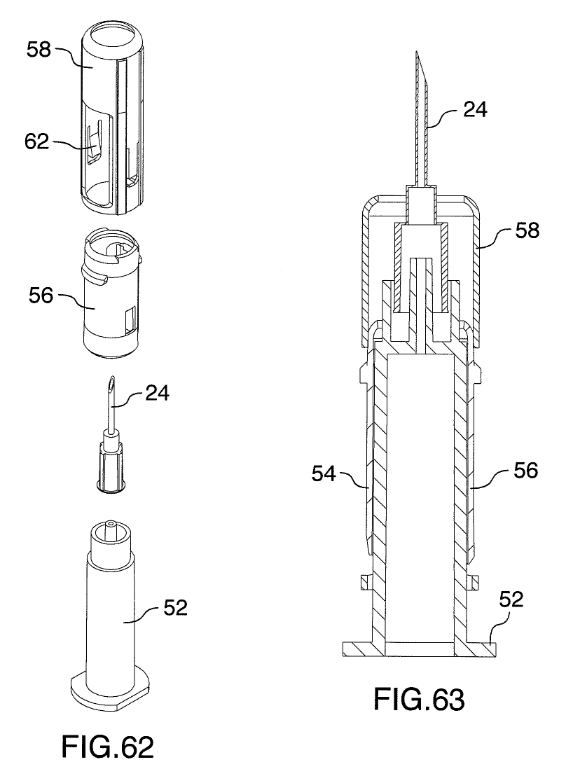

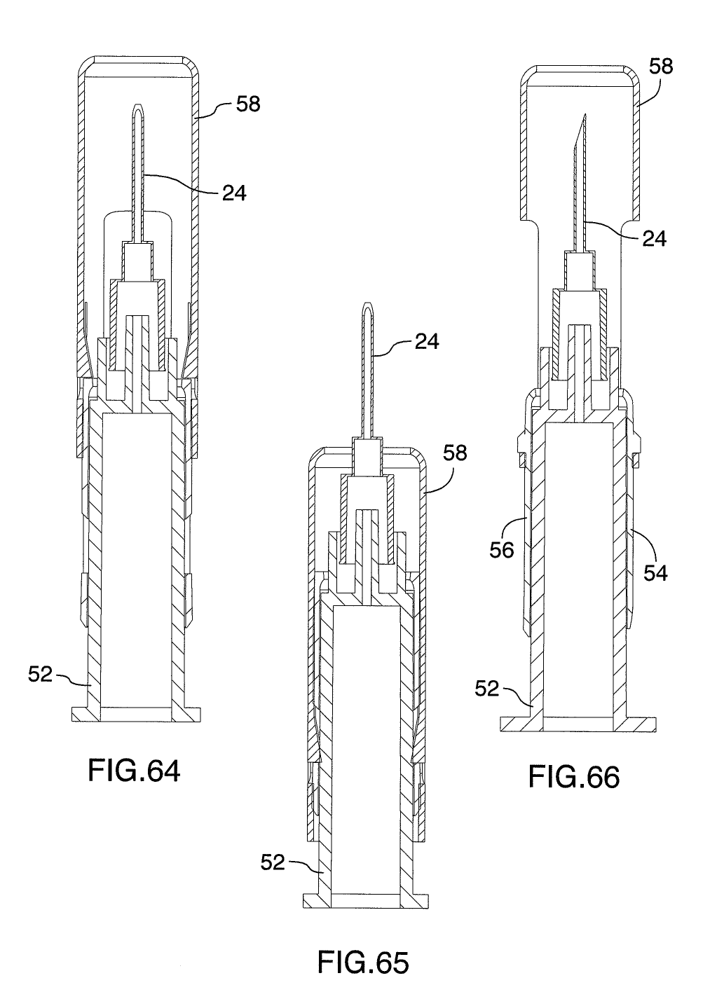

Now referring to FIG. 48, and also to FIG. 49 to 66, there is shown another embodiment of an apparatus 50 for covering a needle 24 of a syringe 52 after use.

In this embodiment, the attaching means of hollow member 56 of the apparatus 50 comprises a single flap 54, as shown in FIG. 50. The apparatus 50 comprises securing members 60 and 62 located on the protective member 58 and which may cooperate with the upper portion of the hollow member 56 to rigidly secure the protective member 58 in the final permanent position once slid.

In this embodiment, as better shown in FIG. 55, the hollow member 56 comprises first and second holding recesses 51, 53 devised to receive and retain the flexible tongues 60, 62 when the protective member 58 is in the operating position. This arrangement helps holding the protective member 58 in place before it is slid in the permanent final position.

The skilled addressee will appreciate that other arrangements may be considered to keep the protective member in place when in the operating position.

For example, it has been contemplated that the protective member be shaped so as to snugly fit on the hollow member.

In a further embodiment, the body of the syringe has a predetermined outer diameter, and the hollow member comprises an inner diameter corresponding to the outer diameter of the body of the syringe. The protective member has an inner diameter corresponding to an outer diameter of the hollow member for providing a snugly fit between the body of the syringe, the hollow member and the protective member.

In one embodiment, the hollow member 56 is made of Polyethylene such as Dow LDPE 722 manufactured by Dow Plastics. Alternatively, various other types of thermoplastic polymer or plastic base substance may be used.

In one embodiment, the protective member 58 is made of polycarbonate, such as Makrolon® 2658 manufactured by Bayer Material Science LLC. Alternatively, various other types of thermoplastic polymer or plastic base substance may be used.

Still in this embodiment, the flap 54 is covered with a release liner such as 3M 4999, an adhesive substance such as 3M 9472 LE. It will be appreciated that in an alternative embodiment, a colored tape may be provided in order to add a visual indication to a user that an adhesive substance is present. The skilled addressee will appreciate that this is of great advantage. In this embodiment, TC 803 Blue Splice Tape may be used. Alternatively, various other types of adhesive substance may be used.

It will be appreciated that the apparatus for covering the needle 24 of the syringe 10 after use disclosed herein is of great advantage since it reduces the risk associated with handling a syringe after use.

It will be appreciated that the apparatus for protecting the needle 24 of the syringe 10 after use disclosed herein is of great advantage since it can be mounted on existing syringes and that it does not require manufacturing a new syringe body for receiving the apparatus.

It will be further appreciated that an advantage of the apparatus disclosed herein is that it can be used on syringes of various diameter sizes and that it can have various size and diameter suitable to cover a given range of syringes.

Indeed, because the hollow member of the apparatus is advantageously attached on the syringe body and not to the finger flange like other prior art devices, the apparatus is suitable on different types of syringe body and needle length. Moreover, a single design of the apparatus may be considered for a specific needle length without being limited to a specific syringe body length.

Although the above description relates to a specific preferred embodiment as presently contemplated by the inventor, it will be understood that the invention in its broad aspect includes mechanical and functional equivalents of the elements described herein.

1. An apparatus for protecting a needle of a syringe after use, the syringe comprising a body, the apparatus comprising:
a hollow member for receiving the body of the syringe, the hollow member comprising attaching means for securing said hollow member to said body of said syringe; and
a protective member slidably mounted on the hollow member and slidable between a syringe operating position wherein said protective member covers at least one part of said body of said syringe and a permanent final position wherein said protective member cover said needle of said syringe.

2. The apparatus of claim 1, wherein said attaching means of said hollow member comprise at least one flap securable to said body of said syringe.

3. The apparatus of claim 2, wherein said at least one flap comprises first and second flaps.

4. The apparatus of claim 3, wherein said first and second flaps are opposite to each other on said hollow member.

5. The apparatus of claim 2, wherein said at least one flap comprises an inner side facing said body of said syringe, said inner side comprising an adhesive substance and a removable protecting layer covering said adhesive substance.

6. The apparatus of claim 5, wherein said adhesive substance comprises a double side tape adhesive.

7. The apparatus of claim 5, wherein said adhesive substance comprises a colored layer for visually indicating presence of the adhesive substance.

8. The apparatus of claim 7, wherein said colored layer comprises a blue layer.

9. The apparatus of claim 1, wherein said hollow member comprises stop means cooperating with the protective member for preventing sliding of the protective member away from the hollow member.

10. The apparatus of claim 1, wherein said protective member comprises at least one securing member cooperating with the hollow member so that the protective member is rigidly secured to the hollow member when said protective member is slid in the permanent final position.

11. The apparatus of claim 10, wherein said at least one securing member comprises first and second securing members, each respectively comprising first and second flexible tongues.

12. The apparatus of claim 11, wherein each of the first and second tongues comprises a projecting member projecting inwards the protective member.

13. The apparatus of claim 11, wherein said hollow member comprises an upper portion having an abutting element cooperating with the flexible tongues.

14. The apparatus of claim 13, wherein said hollow member comprises stop means cooperating with the protective member for preventing sliding of the protective member away from the hollow member.

15. The apparatus of claim 10, wherein said hollow member comprises a holding recess adapted for receiving said securing member therein in the syringe operating position.

16. The apparatus of claim 13, wherein said hollow member comprises first and second holding recesses, each being adapted for receiving a corresponding flexible tongue therein in the syringe operating position.

17. The apparatus of claim 16, wherein said first and second recesses are opposite to each other on said protective member.

18. The apparatus of claim 3, wherein said protective member comprises at least one securing member cooperating with the hollow member so that the protective member is rigidly secured to the hollow member when said protective member is slid in the permanent final position.

19. The apparatus of claim 18, wherein said at least one securing member comprises first and second securing members, each respectively comprising first and second flexible tongues.

20. The apparatus of claim 19, wherein each of the first and second tongues comprises a projecting member projecting inwards the protective member.

21. The apparatus of claim 19, wherein said hollow member comprises an upper portion having an abutting element cooperating with the flexible tongues.

22. The apparatus of claim 21, wherein said hollow member comprises first and second holding recesses, each being adapted for receiving a corresponding flexible tongue therein in the syringe operating position.

23. The apparatus of claim 22, wherein said first and second recesses are opposite to each other on said protective member.

24. The apparatus of claim 23, wherein said first and second flaps are opposed to each other on said hollow member, said flaps and said recesses extending in an alternate configuration.

25. The apparatus of claim 1, wherein said hollow member comprises an upper portion having a supporting surface adapted for resting on an upper surface of the body of the syringe.

26. The apparatus of claim 24, wherein said upper portion of said hollow member has a supporting surface adapted for resting on an upper surface of the body of the syringe.

27. The apparatus of claim 1, wherein said hollow member has an inner diameter corresponding to the outer diameter of said syringe.

28. The apparatus of claim 1, wherein said protective member has a predetermined shape so that the hollow member snugly fits therein.

29. The apparatus of claim 1, wherein the body of the syringe has a predetermined outer diameter, and further wherein said hollow member comprises an inner diameter corresponding to the outer diameter of the body of the syringe, and wherein said protective member has an inner diameter corresponding to an outer diameter of the hollow member for providing a snugly fit between said hollow member and said protective member.

30. The apparatus of claim 1, wherein said hollow member is made of a material selected from a group consisting of thermoplastic polymer and plastic base substance.

31. The apparatus of claim 1, wherein said hollow member is made of polyethylene.

32. The apparatus of claim 1, wherein said protective member is made of a material selected from a group consisting of thermoplastic polymer and plastic base substance.

33. The apparatus of claim 1, wherein said protective member is made of polycarbonate.

34. A method for protecting a needle of a syringe after use, the syringe comprising a body, the method comprising:
providing an apparatus comprising:
a hollow member for receiving the body of the syringe, the hollow member comprising attaching means for securing said hollow member to said body of said syringe; and
a protective member slidably mounted on the hollow member and slidable between a syringe operating position wherein said protective member covers at least one part of said body of said syringe and a permanent final position wherein said protective member cover said needle of said syringe;
inserting said syringe into said hollow member of said apparatus;
securing said attaching means of said hollow member to said body of said syringe; and
after use of said syringe, sliding said protective member in said permanent final position wherein said protective member cover said needle of said syringe.
